(19) United States
(12) Patent Application Publication
(10) Pub. No.: US 2012/0045405 A1
(43) Pub. Date: Feb. 23, 2012

(54) UNDER EYE CREAM

(76) Inventors: Miles E. Gilman, Miami, FL (US); Joseph S. Bertino, JR., Schenectady, NY (US)

(21) Appl. No.: 12/858,560

(22) Filed: Aug. 18, 2010

(51) Int. Cl. A61K 8/18; A61Q 19/02

(52) U.S. Cl. 424/62

(57) ABSTRACT

The present invention relates to an under eye cream for the treatment of swelling, bags, wrinkles, and dark circles for the area underneath the eye.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to an under eye cream. In particular, the present invention relates to an under eye cream for treating the specific swelling, bags, wrinkles, dark circles, and the like associated with the area under the eyes.

2. Description of Related Art

The area underneath the eyes has particular problems not associated with any other part of the skin. While all areas of the skin can experience wrinkles, irritation, and redness, the area under the eye also experiences bagging or drooping that other skin areas do not experience. While some of these problems are related to the aging process, they are also related to rest issues, stress, weight, body fat, and the like and can be temporary in nature though a problem while they last. A search of the internet indicates there are literally thousands of under eye cream preparations for treating these special conditions, but it is also clear that most of them do nothing or at best moisturize the area. While moisturizing reduces some wrinkling, does little or nothing for bagging, dark circles, or severe wrinkling that only occurs under the eyes.

The skin under the eye is very thin and when blood passes though the large veins close to the surface of the skin, it can create a bluish tint to the skin. The more transparent the skin is, the darker the circles will appear. One reason for dark circles is that the capillaries in the area become leaky with the loss of collagen. In addition, red blood cells leak out, with the heme and bilirubin products viewed as dark circles. Hyperpigmentation is a high amount of melanin in the skin and is very often the reason for dark circles under the eyes as well. As the blood vessels in the area under the eye become engorged with blood, the circles become darker. Because the skin color affects transparency, fair skinned people have more of a problem with this trait, however, this is not just a problem of fair skinned people. Other causes for dark circles include fatigue, sun exposure, dehydration, renal (kidney) problems, thyroid problems, certain drugs, and the like which have nothing directly to do with skin color and can occur in everyone.

The area around the eye is typically susceptible to wrinkling and dryness due to the lesser amount of oil glands compared to the rest of the face or other areas of skin adding to the unique skin problems around the eye. Puffiness of eyes and eye bags are different conditions. Fluid retention is the cause of most puffy eye conditions, which is typical in most people in the morning since fluid buildup overnight during sleep is common, baggy eyes, however, are normally the result of an accumulation of fat in the eye area.

Numerous moisturizers, emollients, humectants, and the like are well known in the industry to add moisture to the skin. Other ingredients by themselves are also known to have some limited beneficial value in producing an anti-wrinkle effect. However, the current combinations of such ingredients still do not address all the problems that can happen in this one particular area of the face. A particular composition is disclosed in US published patent application 2008/0081085 to Mitra et al., published Apr. 3, 2008. This application attempts to use several plant extracts to treat this exact problem but is extremely expensive and does not appear to be more effective than other known products. It does appear to have a natural or organic component and for some is preferable even if it is not a better product.

There is a clear need for effective formulations for the treatment of the entire range of under eye problems.

### BRIEF SUMMARY OF THE INVENTION

The present invention relates to the discovery that specific peptides in combination with yeast extract and moisturizer are far more effective in producing a reduction in the symptoms of aging, wrinkling, dark circles, bagging, and the like associated with the area under the eye in humans.

Accordingly, the invention relates to a cream for the treatment of the skin underneath the eye of a human comprising:
a) from about 9% to about 12% percent of one or more moisturizer;
b) from about 3 to about 6% of one or more peptide selected from the group consisting of palmitoyl oligopetide and palmitoyl tetrapeptide-7;
c) from about 0.5 to about 3% saccharomyces lysate extract;
d) a skin whitening agent from about 1% to about 3%; and
e) a cosmetically acceptable vehicle.

### DETAILED DESCRIPTION OF THE INVENTION

While this invention is susceptible to embodiment in many different forms, there is shown and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the phrase “skin underneath the eye” refers to the area from about the edges of the eye associated with crow's feet to the area under the eye associated with bags or dark circles. As used herein “cream” can mean a water or oil soluble cream which can be applied on the skin underneath the eye. One skilled in the art will understand that this is a cosmetic and one can also include an acceptable vehicle for application to this area. While the term cream is used it also includes gels, lotions, or any other topical formulation suitable to apply underneath the eye which is formulated in a cosmetically acceptable vehicle.

As used herein the term “moisturizer” refers to those ingredients that are designed to make the external layers of the skin (epidermis) softer, more pliable, and increase its hydration (water content) by reducing evaporation. Moisturizers are present in the present invention from about 9% to about 12%. Exemplary moisturizers are well known in the art and include Polawax, jojoba oil, long chain hydrocarbons such as squalane, and long chain esters such as isopropyl myristate. In one embodiment the moisturizer consists essentially of squalane and in another embodiment the moisturizer consists essentially of hyaluronic acid. In one embodiment the moisturizer consists of a plurality of moisturizers and in another embodiment consists of a combination of hyaluronic acid and squalane.

One unique aspect of the present invention is the inclusion of at least one peptide selected from the group consisting of palmitoyl oligopeptide and palmitoyl tetrapeptide-7. The peptide comprises from about 3% to about 6% of the composition. In one embodiment the peptide consists of both palmitoyl oligopeptide and palmitoyl tetrapeptide-7, and in another embodiment it consists of the two peptides in equal amounts.

A yeast extract is utilized in the present invention namely from about 0.5% to about 3% of saccharomyces lysate extract. In one embodiment the extract is present in about 1% concentration in the composition. This ingredient acts as a humectant and when used in combination with the peptides of the present invention it also acts to reduce puffiness.

The composition may further include, if desired, a skin conditioner. The skin conditioner may be suitable skin conditioner, as will be appreciated by those skilled in the art. Exemplary skin conditioners include phytantriol, panthenyl ethyl ether, primula veris extract, chamomi, sambucus nigra flower extract, panthenol, polyquaternium-51, cetyl alcohol, glycolic acid, stearyl alcohol, sodium PCA and the like, or combinations thereof. The skin conditioner can be included in the present inventive composition in any suitable amount, but may desirably be included in an amount of from about 0.1% to about 5% by weight of said composition.

Skin whitening agents include n-Hydroxysuccinimide Chrysin and could also include hydroquinones and the like that have skin whitening ability. These compositions can stimulate the enzyme involved in the clearance of bilirubin.

As used herein the expression “emollient” material refers to oleaginous, hydrophobic materials selected from the group consisting of emollient oils; emollient fatty acids, fatty alcohols and fatty acid esters containing a C8-C20 acyl or alkyl group, preferably a C12-C18 acyl or alkyl group; lanolin; cholesterol; hydrophilic lanolin derivatives; phospholipids; and biological extracts. These materials are well known in the art and when deposited upon the skin in a controlled proportion and manner are effective to smooth, soften and moisturize the skin without a greasy after-feel.

Emollient oils include animal oils, vegetable or plant derived oils, hydrocarbon oils, and silicone oils. The oils may be low viscosity, e.g., up to 1000 centipoises (cps.) or high viscosity, e.g., 2000 cps to over 10,000 cps. Specific examples of animal and vegetable or plant derived oils include mink oil, turtle oil, coconut oil, jojoba oil, almond oil, peanut oil, wheat germ oil, rice bran oil, corn oil, soybean oil, olive oil, safflower seed oil, sunflower seed oil, cottonseed oil, apricot kernel oil, peach kernel oil, walnut oil, palm kernel oil, poppyseed oil, hazelnut oil, grapeseed oil, canola oil, avocado oil, macadamia seed oil, castor oil, and mixtures thereof. Specific examples of hydrocarbon oils are mineral oil, paraffin oil, and squalane. Specific examples of silicone oils are polymethylsiloxanes, polymethylphenylsiloxanes, cyclic polysiloxanes, polysiloxanes modified by polyoxyalkylenes or fatty acids or fatty alcohols, and mixtures of the foregoing. Where oils contain unsaturated compounds, use of the hydrogenated version of the oil is preferred.

Specific examples of emollient, hydrophobic compounds containing a fatty (C12-C18) acyl or alkyl group include esters such as isopropyl myristate, isopropyl palmitate, sucrose distearate, butyl stearate, hexyl laurate, capric/caprylic triglyceride, 2-ethylhexyl palmitate, diisopropyl adipate, octyl isononanoate, isopropyl isostearate, isocetyl palmitate, distearyl maleate, diglyceryl diisostearate, and mixtures thereof. Specific examples of higher C12-C18 fatty alcohols include cetyl alcohol, stearyl alcohol, oleyl alcohol, and mixtures of the foregoing. Specific examples of C12-C18 fatty acids include myristic acid, palmitic acid, behenic acid, stearic acid, oleic acid, isostearic, and mixtures of the foregoing. Specific examples of hydrophobic, emollient extracts include shea butter or butyrospermum parkii that is a fat derived from karite tree.

Other suitable emollient materials include wax esters, e.g., lanolin; cholesterol and lanolin alcohols; hydrophilic lanolin derivatives, e.g., ethoxylan; and phospholipids, e.g., lecithin and cephalin. Usually any emollient waxes are present only in small amounts.

The emollient material can be comprised of an emollient oil. Preferably, the oil will be mixed with one or more of an emollient compound selected from the group consisting of fatty acids, fatty alcohols, and fatty esters containing a C8-C20 acyl and C8-C20 alkyl fatty group; shea butter; lanolin or a lanolin derivative; and lecithin; with the oil being present in major proportion in the mixture.

The compositions of the invention comprise a cosmetically acceptable vehicle to act as a diluent, dispersant, or carrier so as to facilitate distribution and uptake when the composition is applied to the skin. Vehicles other than, or in addition to, water can include liquid or solid emollients, solvents, humectants, thickeners, and powders.

The cosmetically acceptable vehicle will usually form from 5% to 99.9%, preferably from 25% to 80% by weight of the composition and can, in the absence of other cosmetic adjuncts, form the balance of the composition.

The compositions may be in the form of aqueous, aqueous/alcoholic, or oily solutions; dispersions of the lotion or serum type; anhydrous or lipophilic gels; emulsions of liquid or semi-liquid consistency which are obtained by dispersion of a fatty phase in an aqueous phase (ONV) or conversely (W/O); or suspensions or emulsions of smooth, semi-solid, or solid consistency of the cream or gel type. These compositions are formulated according to the usual techniques as are well known to this art.

Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone), and fluoro oils (perfluoropolyethers). Fatty alcohols, fatty acids (stearic acid), and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols, and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate, and isostearyl palmitate. Acceptable tribasic acid esters include triisopropyl trilinoleate and trilauryl citrate. Acceptable straight chain fatty esters include lauryl palmitate, myristyl lactate, oleyl eurcate, and stearyl oleate. Preferred esters include coco-caprylatelcaprate (a blend of coco-caprylate and coco-caprate), propylene glycol myristyl ether acetate, diisopropyl adipate, and cetyl octanoate.

Suitable fatty alcohols and acids include those compounds having from 10 to 20 carbon atoms. Especially preferred are compounds such as cetyl, myristyl, palmitic, and stearyl alcohols and acids.

Among the polyols which may serve as emollients are linear and branched chain alkyl polyhydroxyl compounds. For example, propylene glycol, sorbitol, and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene, and isoparaffins.

The compositions of the invention may also contain additives and adjuvants which are conventional in the cosmetic, pharmaceutical, or dermatological field, such as hydrophilic or lipophilic gelling agents, hydrophilic or lipophilic active agents, preservatives, antioxidants, solvents, fragrances, fillers, bactericides, odor absorbers and dyestuffs, or colorants. The amounts of these various additives and adjuvants are those conventionally used in the field and, for example, range from 0.01% to 10% of the total weight of the composition. Depending on their nature, these additives and adjuvants may be introduced into the fatty phase or into the aqueous phase.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin, and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate, and mixtures thereof.

Other adjunct components may also be incorporated into the cosmetic compositions. These ingredients may include coloring agents, opacifiers, and perfumes. Specifically, these ingredients may include cosmetically suitable additives such as deionized water, hydrolyzed glycosaminoglycan, sodium hyaluraonate, triethanolamine, propylene glycol, methylparaben, propylparaben, acrylates, C10-C20 alkyl acrylate crosspolymers, C12-C15 alkyl benzoate, panthenol, biotin, sodium chloride, sodium phosphate, and the like. Amounts of these other adjunct components may range anywhere from 0.001% up to 20% by weight of the composition.

A variety of surfactants may be employed in the compositions of the invention. These surfactants are well known to formulation chemists skilled in the art. Surfactants may be cationic, nonionic, or anionic in nature. Preferably, nonionic surfactants are employed in the present microemulsions because they are nonirritating to skin. Anionic surfactants may also be employed, but care should be taken to make the resulting compositions as nonirritating as possible. Suitable surfactants include esters of aliphatic carboxylic acids having from 12 to 18 carbon atoms in combination with either a polyoxyethylene (4-20) sorbitan ether or other sorbitan ether substance. Also, esters of unsaturated carboxylic acids having from 12 to 18 carbon atoms together with either a polyoxyethylene (4-20) sorbitan ether or other sorbitan ether substance may be employed as surfactants. Finally, combinations of the aliphatic and unsaturated carboxylic acid esters described above may be employed in combination.

The compositions of the invention may also contain one or more co-surfactants in addition to the surfactants described above. Exemplary co-surfactants suitable for use herein include fatty aliphatic alcohols having from 12 to 18 carbon atoms, such as cetyl alcohol or myristyl alcohol, short chain alkyl alcohols having from 1 to 3 carbon atoms with or without branching, or mixtures of these two ingredients. Typically, long chain alkyl alcohol co-solvents are employed in water-in-oil microemulsions.

A variety of skin compatible oils may also be employed in a composition of the invention which are known and commonly used by those of ordinary skill in the art. Exemplary oils include hydrocarbon straight chain alkyl compounds having from 12 to 18 carbon atoms, hydrocarbon branched alkyl compounds having from 12 to 30 carbon atoms, esters of straight alkyl chain hydrocarbons having from 12 to 18 carbon atoms together with a fatty alcohol having from 3 to 14 carbon atoms or a vegetable oil containing mono, di- or triglycerides, and cyclic dimethyl polysiloxane compounds such as cyclomethicone.

The combinations of the invention will also contain one or more suitable skin compatible humectants. These humectants are polar in nature and include deionized water, propylene glycol, glycerine, sorbitol, or other polyhydric alcohols.

All cosmetic compositions must be protected against the growth of potentially harmful microorganisms, and therefore preservatives are added as a routine. While it is in the aqueous phase that microorganisms tend to grow, microorganisms can also reside in the oil phase. As such, preservatives which have solubility in both water and oil are preferably employed in the present compositions. Generally from one tenth of one percent by weight to one percent by weight of preservatives are adequate. The traditional preservatives for cosmetics and pharmaceuticals are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives for a preferred emulsion product of this invention are methyl and propyl para-hydroxybenzoates, imidazolidinyl urea, and quaternium-15. The preservatives should be selected having regard for the use of the composition and possible incompatibilities between the preservatives and the other ingredients in the emulsion.

The compositions of the invention may be prepared by procedures well known to formulation chemists. Typically, an oil phase is prepared by combining all hydrophobic components, as well as the water-insoluble solids, in a container and heating the resulting mixture under agitation until all the ingredients are dissolved. In a separate container, an aqueous phase is prepared by combining all of the hydrophilic components, as well as the oil-insoluble solids, and heating the resulting mixture under constant stirring until the mixture is homogeneous. The surfactants and cosurfactants are separately combined and mixed until homogeneous, and heat may be applied if necessary. The three phases thus prepared are combined and stirred until homogeneous and the solution becomes clear when allowed to stand at room temperature. The composition is finally allowed to stand for approximately 24 hours in order for the composition to achieve equilibrium. The composition may be transferred to appropriate containers for storage until needed for application to individuals in need of a composition of the invention.

The following Examples illustrate formulations of the invention, and methods for their preparation. The Examples are not intended to be limiting to the scope of the present invention in any respect and should not be so construed.

### EXAMPLE 1

### Under Eye Cream

| Ingredient | % (w/w) |
|---|---|
| Purified water | 67.0% |
| Squalane | 10.0% |
| Arnica Montana Extract | 3.0% |
| Glycerin | 2.0% |
| Propylene glycol | 2.0% |
| Sodium PCA | 2.0% |
| Steareth 20 | 2.0% |
| n-Hydroxysuccinimide Chrysin | 2.0% |
| Palmitoyl oligopeptide | 2.0% |
| Palmitoyl terapepetide-7 | 2.0% |
| Sacchromyces Lysate Extract | 1.0% |
| Laureth-7 | 1.0% |
| C13-14 Isoparaffin | 1.0% |
| Polyacrylamide | 1.0% |
| Hyaluronic acid | 1.0% |
| Phenoxyethanol | 0.5% |
| Ethylhexyglycerin | 0.5% |
| Total | 100.0% |

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like, apparent to those skilled in the art still fall within the scope of the invention as claimed by the applicant.

What is claimed is:

1. A cream for the treatment of the skin underneath the eye of a human comprising:
a) from about 9% to about 12% percent of one or more moisturizer;
b) from about 3 to about 6% of one or more peptide selected from the group consisting of palmitoyl oligopeptide and palmitoyl tetrapeptide-7;
c) from about 0.5 to about 3% saccharomyces lysate extract;
d) a skin whitening agent from about 1% to about 3%; and
e) a cosmetically acceptable vehicle.

2. A cream according to claim 1 wherein the moisturizer is squalane.

3. A cream according to claim 1 wherein the moisturizer is hyaluronic acid.

4. A cream according to claim 1 which further comprises a humectant.

5. A cream according to claim 4 wherein the humectants is selected from the group comprising glycerin and propylene glycol.

6. A cream according to claim 1 which further comprises an emollient.

7. A cream according to claim 6 wherein the emollient is selected from the group comprising arnica Montana extract and C13-14 isoparaffin.

8. A composition according to claim 1 which further comprises a skin conditioner.

9. A composition according to claim 8 wherein the skin conditioner is sodium PCA.

10. A composition according to claim 1 wherein the composition comprises both palmitoyl oligopeptide and palmitoyl terapeptide-7.

11. A composition according to claim 1 wherein the skin whitening agent is n-hydroxysuccinimide chrysin.
